(19) United States
(12) Patent Application Publication
(10) Pub. No.: US 2021/0052855 A1
(43) Pub. Date: Feb. 25, 2021

(54) HYBRID ELECTROMAGNETIC DEVICE FOR REMOTE CONTROL OF MICRO-NANO SCALE ROBOTS, MEDICAL TOOLS AND IMPLANTABLE DEVICES

(71) Applicant: BIONAUT LABS LTD., Herzliya (IL)

(72) Inventors: Alex KISELYOV, San Diego, CA (US); Michael SHPIGELMACHER, Los Angeles, CA (US); Eran OREN, Tel-Aviv (IL); Be'eri Berl KATZNELSON, Kiryat Tivon (IL); Suehyun CHO, Los Angeles, CA (US); John CAPUTO, Los Angeles, CA (US); Eli VAN CLEVE, Los Angeles, CA (US); Eldad ELNEKAVE, Tel-Aviv (IL)

(73) Assignee: BIONAUT LABS LTD., Herzliya (IL)

(21) Appl. No.: 17/052,132

(22) PCT Filed: May 2, 2019
(86) PCT No.: PCT/US2019/030355

§ 371 Date: Oct. 30, 2020

Related U.S. Application Data
(60) Provisional application No. 62/666,525, filed on May 3, 2018.
(60) Provisional application No. 62/754,893, filed on Nov. 2, 2018.

(51) Int. Cl. A61M 25/01; A61M 25/00; A61B 10/02

(52) U.S. Cl. CPC A61M 25/0158; A61M 25/0045; A61M 2025/09141; A61M 25/0021; A61B 10/02; A61M 25/0108

(57) ABSTRACT

An insertion and retraction device is described for delivery and removal of a microparticle from target tissue. The device comprises a magnetic or magnetizable needle or cannula having a distal end, a proximal end and a lumen adapted to convey microparticles; a tubular catheter receiving the needle or cannula; a pressure device adapted for delivery of microparticles through the needle or cannula lumen by pressure; a magnetic field modulator adapted to move the needle or cannula by modulation of a magnetic field; and a magnetic sensor positioned toward the distal end of the tubular catheter responsive to a magnetic moment of the microparticle.

### FIELD OF THE INVENTION

An insertion and retraction device is described for delivery and removal of a microparticle to/from target tissue.

### BACKGROUND OF THE INVENTION

Devices have been developed to allow for localized drug release via drug carrying elements having nanometer- to millimeter-scale dimensions. Such elements may be referred to as microbots, nanobots or simply as micro-/nano-particles. In the emerging field of miniature robotics, multiple applications require the use of an insertion and retraction device, for delivery via different routes including oral application, injection to the blood stream, catheter, etc. The micro/nanobots need to be accurately inserted and/or removed from the body. Ideally, such devices should be minimally invasive and enable the positioning of at least one miniature robot at a time.

Microfluidics devices equipped with an injection port and exhibiting magnetic or magnetizable properties and “on/off” control switch for controlled microparticle release are described in <<https://m.smiths-medical.com/products/infusion/syringe-infusion/micro-fluid-delivery-system/mangum-micro-fluid-delivery-system>>, which is incorporated by reference.

The following references, which are incorporated by reference, describe medical equipment suitable for microparticle delivery which may be adapted for use with embodiments of the invention. <<http://www.vascularperspectives.com/Cardiology/Microcatheters/ASAHI-Caravel.htm>>; <<https://www.cookmedical.com/products/di_mcs_webds/>>; <<https://www.researchgate.net/figure/FineCross-micro-catheter_fig10_304779104>>.

Biocompatible coatings that would be known to those of ordinary skill in the art are disclosed in Ulery, B. T. et al. “Biomedical Application of Biodegradable Polymers,” J. Polym. Sci. B., Polym. Phys. 2011, 49(12), 832-864, which is incorporated by reference.

Biocompatible structural polymers that would be known to those of ordinary skill in the art are described in Maitz, M. F., et al. “Application of Synthetic Polymers in Clinical Medicine,” Biosurface and Biotribology 2015, 1(3), 161-176, which is incorporated by reference.

A laparoscopic needle that may be adapted for use with the invention is taught in <<http://www.ip.mountsinai.org/blog/magnetic-needle-retriever/>>, which disclosure is incorporated by reference. Similarly, a combination of the proposed therapeutic microparticle and appropriate collection device including but not limited to a magnetizable or magnetic needle could be illustrated by the microsuturing protocol described in <<https://barberneedles.com/blog/?p=116>>, which is incorporated by reference.

Recording moduli including electrodes non-interfering with a microparticle's magnetic moment, are exemplified in <<https://www.researchgate.net/figure/On-the-left-side-the-first-generation-1-magnet-map-1-M-catheter-is-shown-The-second_fig1_6584012>> which is incorporated by reference. These may be adapted for use with the invention.

A representative example of a delivery device that could both accommodate a microparticle and provide dual imaging/navigation information is summarized in Park, J., et al. “Biopsy Needle Integrated with Electrical Impedance Sensing Microelectrode Array towards Real-time Needle Guidance and Tissue Discrimination,” Sci. Rep. 2017, doi:10.1038/s41598-017-18360-4, which is incorporated by reference.

Shape memory materials that may be used in connection with the claimed invention, are described for example in Hanawa, T. “Materials for Metallic Stents,” J. Artificial. Org. 2009, 12(2), 73-79, which is incorporated by reference.

Becker, T. A., et al. “Calcium alginate gel: a biocompatible and mechanically stable polymer for endovascular embolization,” J. Biomed. Mater. Res. 2011, 54(1), 74-86, which is incorporated by reference, describes materials that can be used to prevent the microparticle from being released as it is sheathed into a tube and when it is pulled out.

### SUMMARY OF THE INVENTION

A device according to the invention allows for the administration or insertion of a magnetic particle to the body of human patient or an animal and exhibits the following properties:

1. Reaches a point in the body that is in the vicinity, adjacent to or in an organ or tissue of interest;

2. Provides sufficient inherent and operational safety;

3. Sets the microparticle at a specified point;

4. Provides reliable and reproducible control over the position of microparticle prior to start of propulsion and after payload release;

5. Exhibits a retrieving mechanism that allows for collection and retrieval of a microparticle from the body;

6. Accommodates specific matrices and therapeutic agents including but not limited to powders, liquids, gels that are embedded in a microparticle; representative examples of adjuvant substances appropriate for the procedure are exemplified by but not limited to antiseptic, antibacterial, anti-inflammatory, analgesic, coagulation modulators;

7. Integrates with other modules of the platform including but not limited to magnetic propulsion of the magnetic particles and multiple imaging platforms as exemplified but not limited to ultrasonography, fluoroscopy, MRI; and

8. Can be manipulated using a manual, semi-automated, or completely automated mechanism.

Thus, in one aspect, the invention is embodied as an insertion and retraction device for delivery and removal of a microparticle to/from target tissue, comprising: a magnetic or magnetizable needle or cannula 14 having a distal end, a proximal end and a lumen adapted to convey microparticles; a tubular catheter 12 receiving the needle or cannula; a pressure device 18 adapted for delivery of microparticles through the lumen by pressure; a magnetic field modulator 16 adapted to move the needle or cannula by modulation of a magnetic field; and a magnetic sensor 13 positioned toward the distal end of the tubular catheter responsive to a magnetic moment of the microparticle.

In another aspect, the invention is embodied as a system for delivery and/or retrieval of a microparticle from target tissue, comprising: at least one microparticle 22 having a magnetic moment, immobilized in a plug of biocompatible and biodegradable polymer 21; a magnetic or magnetizable needle or cannula 14 having a lumen adapted receive and release the at least one microparticle in said plug of biocompatible and biodegradable polymer; a tubular syringe or catheter 14 adapted to receive the needle or cannula; a pressure device 18 adapted for delivery of the microparticle through the lumen by pressure; a magnetic field generator 16 adapted to control movement of the needle or cannula in the syringe or catheter; and a magnetic sensor 13 positioned toward the distal end of the tubular catheter responsive to the magnetic moment of the microparticle and operatively connected to the magnetic field generator.

In another aspect, the invention is embodied as an insertion and retraction device for tissue biopsy, comprising: a magnetic or magnetizable needle or cannula having a distal end, a proximal end and an opening on the distal end adapted to receive a biopsy sample; leaflets 41 at the distal end of the needle or cannula adapted to open and close the opening; a rod 51 adapted to be actuated to open and close the leaflets; a magnetic field modulator adapted to move the needle or cannula by modulation of a magnetic field; and a magnetic sensor positioned toward the distal end of the tubular catheter responsive to a magnetic moment of the device. In this embodiment, the needle may comprise, partly or entirely, a shape memory metal alloy including nickel titanium alloys commonly referred to as Nitinol. Laser cutting a tube of an appropriate size and using a jig ensures that the tube tip may be formed into a desired tip shape.

Thus, an insertion and retraction device according to embodiments of the invention a comprises a magnetic or magnetizable needle or cannula having a distal end, a proximal end and an opening on the distal end adapted to receive a biopsy sample.

Leaflets at the distal end of the needle or cannula are adapted to open and close the opening. A rod positioned inside the needle or cannula permits actuation of the leaflets to open and close the opening. A magnetic field modulator is adapted to move the needle or cannula by modulation of a magnetic field and a magnetic sensor positioned toward the distal end of the tube is responsive to a magnetic field or gradient near distal end vicinity, in order to move the tube in the right direction. For example, a magnetic sensor at the end of the tube can sense the location of a magnetic microparticle in the vicinity of the distal end of the tube (providing a measure of the magnetic field gradient). This signal can in turn be used to move the distal end of tube along the magnetic field gradient vector towards the microparticle, to allow for collection of microparticle.

### BRIEF DESCRIPTION OF THE DRAWINGS

The subject matter regarded as the invention is particularly pointed out and distinctly claimed in the concluding portion of this specification. The invention, however, both as to organization and method of operation, together with objects, features, and advantages thereof, may best be understood by reference to the following detailed descriptions when read with the accompanying drawings in which:

FIG. 1 depicts a magnetic needle with a switchable magnetic field via a permanent electro holding magnet inserted through a syringe tube, according to embodiments of the invention;

FIG. 2 depicts delivery of a microparticle with a delivery device in a matrix of biodegradable polymer gel, according to embodiments of the invention;

FIG. 3 depicts a steerable needle tip with a control handle for searching different positions and orientations in trying to retrieve a microparticle, according to embodiments of the invention;

FIG. 4 depicts a magnetic microbiopsy device designed for delivery and retraction of microparticles according to embodiments of the invention;

FIG. 5A, FIG. 5B, FIG. 5C and FIG. 5D depict different positions of a delivery and retraction device according to embodiments of the invention, including (in FIG. 5A) an open configuration suitable for the particle collection, and (in FIG. 5B, FIG. 5C and FIG. 5D) a closed configuration suitable for delivery or retraction of the collected microparticle; and

FIG. 6A, FIG. 6B and FIG. 6C illustrate operation of the device according to an embodiment of the invention.

The Figures are illustrative, and the use of reference numerals herein should not be deemed to limit the invention to specific embodiments. The Figures are not necessarily drawn to scale and features that are not necessary for an understanding of the invention described have been omitted.

### DETAILED DESCRIPTION OF THE INVENTION

In the following detailed description, numerous specific details are set forth in order to provide a thorough understanding of the invention. However, it will be understood by those of ordinary skill in the art that the present invention may be practiced without these specific details. In other instances, well-known methods, procedures, and/or components have not been described in detail so as not to obscure the present invention.

According to some embodiments of the invention, a magnetic or magnetizable, electrostatic or pneumatic needle/catheter is made of biocompatible metal or metal alloys exemplified by surgical steel, Ti-, Mg-, Co-, Cr-alloys, their respective composite materials and others. In embodiments of the device, a specific surgical, diagnostics or alternative medical device, probe, catheter or needle may be elaborated upon to accommodate the microparticle, the microparticle in a plug or alternative administration matrix. In a representative example, an external pressure device, exemplified by but not limited to, a microsyringe, a microreservoir, micropump or alternative external or implantable microfluidics device is equipped with injection port exhibiting magnetic or magnetizable properties and “on/off” control switch for a controlled microparticle release, as described in <<https://m.smiths-medical.com/products/infusion/syringe-infusion/micro-fluid-delivery-system/mangum-micro-fluid-delivery-system>>, which is incorporated by reference.

The device may feature a specific coating that includes but is not limited to Cr, Pt, Pd, Au or other biocompatible metal and/or metal alloy coatings to ascertain safety and delivery of a microparticle. In a representative example, said device may exhibit parameters similar to the examples described in the following references (which are incorporated by reference) <<http://www.vascularperspectives.com/Cardiology/Microcatheters/ASAHI-Caravel.htm>>; <<https://www.cookmedical.com/products/di_mcs_webds/>>; and <<https://www.researchgate.net/figure/FineCross-micro-catheter_fig10_304779104>>,in each case adapted according to the level of ordinary skill in the art to exhibit specific magnetic properties and coating in the operational area as discussed above.

In embodiments, the device may exhibit specific polymer coating that include but not limited to a variety of biocompatible polymers including but not limited to polylactides, polyglycosides, poly(Lactide-co-glycoside), poly(hydroxyalcanoates), perfluorinated or partially fluorinated polymers, polycaprolactones, polypropylene fumarate, polyanhidrides, polyacetals, polycarbonates, polyurethanes, polyphosphazenes and combination polymers (Ulery, B. T. et al. “Biomedical Application of Biodegradable Polymers,” J. Polym. Sci. B., Polym. Phys. 2011, 49(12), 832-864).

In embodiments, the device may be made of a suitable biocompatible polymer, cross-linked polymers, copolymers or polymers grafted with diverse materials include carbon or metal fibers as exemplified but not limited to PE, PP, PTFE, PVC, PMMA, pHEMA, dacron, PGA, PLGA, PLLA, PDLA, PDO, PEEK, PES, Polyurethane, (Maitz, M. F., et al. “Application of Synthetic Polymers in Clinical Medicine,” Biosurface and Biotribology 2015, 1(3), 161-176)

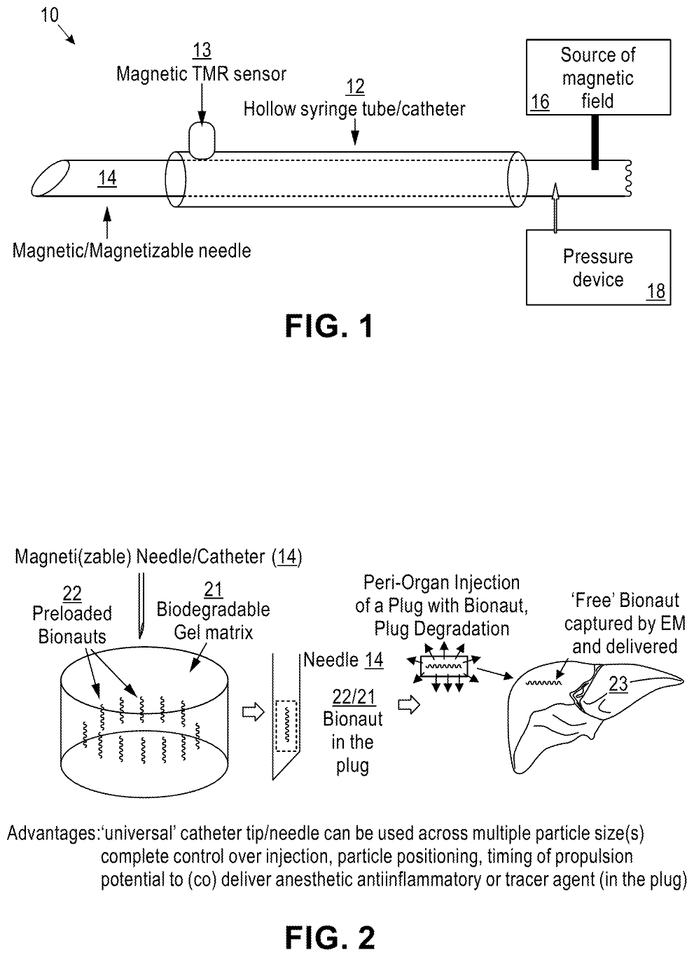

In embodiments, as shown in FIG. 1, a device 10 according to the invention comprises a hollow tube such as syringe 12, including but not limited to a narrow needle, cannula or catheter (referred to herein as a tubular catheter) to reach the point of interest. In a representative example, it may include a hollow tube made of a biocompatible metal as described above. The tubular 12 is inserted into the operational area via a predetermined path that minimizes damage or other risks, and positions it in the proximity of, or into, the organ or tissue of interest. Through this tube, materials can be injected by applying a mechanical force, using gas-, liquid-pressure, electrical, electromagnetic, magnetic, acoustic, vibrational or optical stimuli. In a representative example, the retrieval device is fit within the surgical instrument as represented by the laparoscopic needle <<http://www.ip.mountsinai.org/blog/magnetic-needle-retriever/>>. Similarly, a combination of the proposed therapeutic microparticle and appropriate collection device including but not limited to a magnetizable or magnetic needle could be illustrated by the microsuturing protocol described in <<https://barberneedles.com/blog/?p=116>>.

Still referring to FIG. 1, in a representative embodiment of this invention, setting of the microparticle can be performed by using a magnetic or ferromagnetic element such as a needle 14 that can be loaded with the microparticle beforehand and inserted through the aforementioned syringe or hollow tube 12, or alternatively set with the microbot when already in the needle. In embodiments, release of the microparticle is performed by manipulating a localized source of magnetic field 16 with mechanisms including a permanent electro holding magnet that switches an external magnetic component ‘off’ by countering its magnetic moment with an opposite field from an electromagnetic coil. The field from the external permanent electro holding magnet is led through a ferromagnetic needle 14 inside the syringe, and thus attracts the magnetic microparticle. When the needle is pushed all the way through the syringe and the microparticle is in the desired location, the effective magnetic field is turned off and the lack of magnetic attraction allows the microparticle to stay in place, while the ferromagnetic needle is being pulled away.

Alternatively, the system may comprise a permanent magnetic needle using the friction created between the microparticle element and the tissue when it is unsheathed and sheathed into the external syringe cover. Additional options include the use of the syringe itself as the magnetic material as exemplified but not limited to Cr-, W-, Mo-, Ni- or Ni-alloys.

In another embodiment, as shown in FIG. 2, the micro-/nanoparticle 22 may be delivered to the proximity of, or inside of, an organ 23 or tissue as a treatment immobilized in a plug of biocompatible and biodegradable polymer 21 to a) secure microparticle positioning, b) ascertain controlled and timed release of a microparticle from the plug upon initiation of external propulsion stimuli as exemplified by magnetic or electromagnetic, ultrasound, acoustic, electrical or optical stimuli, c) provide specific imaging enhancement for the device, its specific compartment (ex., tip), polymer plug and particle initial positioning, as exemplified but not limited by co-administration of a contrasting agent in a plug (iodinated, complexed Hf, microbubbles) and d) deliver adjuvant therapeutic agents as exemplified but not limited to exemplified by but not limited to antiseptic, antibacterial, anti-inflammatory, analgesic, immunomodulators, coagulation modulators and other co-administered agents. In a representative manifestation of this invention, the catheter featuring magnetizable needle may also contain additional recording moduli including electrodes non-interfering with microparticle's magnetic moment, as exemplified in <https://www.researchgate.net/figure/On-the-left-side-the-first-generation-1-magnet-map-1-M-catheter-is-shown-The-second_fig1_6584012>>.

In an embodiment, another physical device is used for retrieving the microbot when its use inside the patient's body is over. The retrieving device typically resembles the insertion device and utilizes a hollow syringe as described above to position its tip in the predetermined volume to collect the microparticle. The predetermined volume could be designated using both external imaging platforms including but not limited to ultrasound, fluoroscopy, MRI a combination of thereof, suitable fiduciary markers as exemplified but not limited to preinjected Au particles or iodinated materials. Both propulsion and navigation of the microparticle into the designated collection area could be mediated by aforementioned external stimuli and imaging methods. In the preferred embodiment of this invention, the particle is propelled by external magnetic or electromagnetic field and imaged using conventional ultrasonic imaging equipment. A collection moiety, as exemplified by the needle tip could be navigated into the collection area using similar imaging modalities.

The system can be used in conjunction with means to aid in positioning of the syringe in the correct position to increase the fidelity of both insertion and retraction. The syringe may include additional means to increase its visibility in commonly used medical imaging devices such as X-ray imaging, ultrasound imaging, CT and others. For example, the syringe may include a radio-opaque element with scribing that allows easy recognition and orientation inside a patient's body by using X-ray imaging. Alternatively, or in addition, the syringe may include an ultrasound reflector element to increase the visibility and ease of use in conjunction with an ultrasound device. Alternatively, a syringe may include a vibrating MEMS mechanism enhancing ultrasound visibility. A representative example of a delivery device that could both accommodate said microparticle and provide dual imaging/navigation information is summarized in Park, J., et al. “Biopsy Needle Integrated with Electrical Impedance Sensing Microelectrode Array towards Real-time Needle Guidance and Tissue Discrimination,” Sci. Rep. 2017, doi:10.1038/s41598-017-18360-4.

The system of FIG. 1 can be used with a magnetic sensor 13 that corresponds to the magnetic moment of the microparticle. The magnetic sensor is either located at the tip of the syringe, or in the vicinity of the tip, or externally with a mechanism that guides the magnetic field lines from the microparticle to an external sensor. Typical sensors require high resolution and detection of a low magnetic field. Relevant sensors include among others, tunnel-magnetoresistance (TMR) sensors, giant magnetoresistance (GMR) sensors, and superconducting quantum interference device (SQUID) sensors in case of an external use and others.

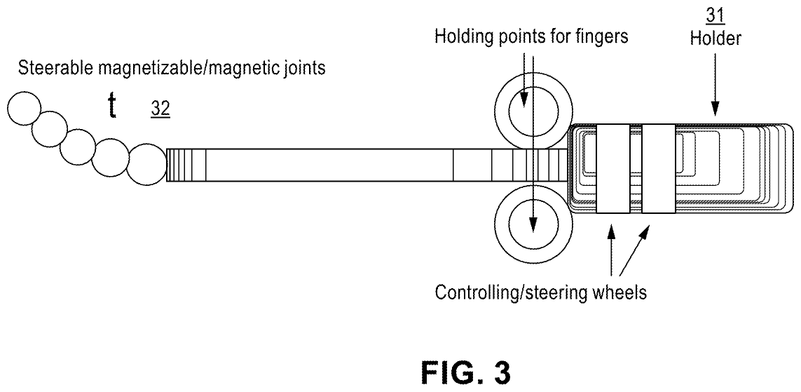

In a representative embodiment, the operational diameter of the delivery needle or catheter may vary between 100 μm and 2,000 μm. The tip of the magnetic needle can be of a static configuration, meaning simply inserted to a single point. As shown in FIG. 3, it can also be made of a steerable element, which can typically be moved and controlled with an error margin of a few millimeters. For example, it can be steered using fibers that are connected to an external handle 31 and which are capable of manipulating the needle's tip's position 32 in a sphere thus allowing moving of typically 3-5 mm to each side. Steering the tip of the needle can compensate for different particle orientations and discrepancies in location that can affect the magnetic field strength and thus the attractive forces between the microparticle and the magnetic needle.

When the tip is positioned in the vicinity of the microparticle, specifically within 1-2 microparticle sizes (L=250-5,000 μm), a magnetic field is applied to attract and collect the microparticle. This can be performed in a manner similar to that described above, with an internal needle unsheathed next to the microparticle followed by application of a switchable magnetic field from an external source, or alternatively by using a permanent magnet needle, or a permanent magnet tip for a non-magnetic needle.

In another embodiment, the retrieval process is performed by using the same device to reduce or eliminate the need for extraction and additional insertion of the syringe after the initial placement of the microparticle. This option is particularly relevant when a medical risk prevents perforation of multiple points for the purpose of inserting and retrieving the microparticle. In this case, after the syringe was inserted and its tip was positioned in the point of interest, the magnetic needle can be left in place and be used in the same manner when the microparticle returns to the same point. Alternatively, it can be pulled out to avoid the presence of any ferromagnetic material during an operation which might utilize external magnetic fields. The magnetic needle can be placed back once the operation has ended and there is no additional magnetic field applied.

In some embodiments, additional means can be utilized to increase the probability of retrieving the microparticle. In a representative example, the microparticle capture can be mediated by a mechanical device as exemplified by (micro)tweezers or a (micro)mesh that could be deployed via said needle/syringe and comprised of a memory material as exemplified by but not limited to metal, metal composite, polymer and or polymer composite materials (ex., Hanawa, T. “Materials for Metallic Stents,” J. Artificial. Org. 2009, 12(2), 73-79). Alternatively, a super-elastic element, such as Nitinol, can be used to open and close on the microparticle by sheathing and unsheathing from an external tube. In all of these options, the particle capture is performed in addition to a magnetic element that aids in positioning the microparticle in the desired location. Furthermore, adhesion mediated by specific biocompatible coating agents exemplified but not limited to alginate gels or respective composite material and co-polymers (Becker, T. A., et al. “Calcium alginate gel: a biocompatible and mechanically stable polymer for endovascular embolization,” J. Biomed. Mater. Res. 2011, 54(1), 74-86) can be used to prevent the microparticle from being released as it is sheathed into a tube and when it is pulled out. A representative example of the larger-scale device that could be amended to accommodate a said delivery and retraction of microparticle using combined magnetic and mechanical means is included <<https://www.amazon.com/Elitexion-Flex-Cable-Mechanic-Pick-Magnet/dp/B011WHC34K>>.

Additional means of collecting the microparticle can be introduced by using a suction element such as a vacuum pump or a syringe used to create low pressure relative to the pressure in the vicinity of the microparticle. In this case, a small tube will be introduced, typically having smaller size than the microbot, and low pressure will enable attraction forces to increase probability of retraction.

The magnetic element can be of an inflatable type, meaning a balloon-like device at the tip of the syringe, which can be inflated to present a larger surface area for the attraction forces. Typically, in such a case, the balloon can be inflated with a ferro-fluid to allow magnetic properties.

An additional mechanism for proper positioning of the device prior to insertion/retraction may involve a robotic arm holding the device, receiving input from a control system. The control system can estimate the location of the injection/retraction device in relation to the target area and place the injection/retraction device at the right position outside of the patient/animal body prior to tissue penetration, in order to accurately approach the target area. Alternatively, the positioning of the injection/retraction device can be done manually, based on visual feedback from the control system presented to the human operator in the form of clear steering commands (e.g., move left/right/up/down, rotate X degrees).

The device is typically used by medical experts but can also be used in conjunction with automated systems such as actuators, motors, robotic arms and with different imaging capabilities such as ultrasound, x-ray, optical cameras and others.

In another embodiment, a device for mechanical insertion and removal of a microparticle from patient's body mediates introduction of a syringe or a catheter that performs a biopsy-like procedure that cuts and grabs a tissue segment, of typical sizes of 1-5 mm in each dimension, for the purpose of retrieving the microparticle within the tissue.

In this embodiment, insertion of the microparticle can be similar to the one presented above, but retraction is performed using a mechanical element, typically made of metal with sharp edges that allows cutting a piece of tissue and inserting it to a hollow tube.

Cutting can typically be performed by unsheathing scalpel-like metal elements on several side, thus cutting around the desired tissue segment followed by scooping the resulting microbiopsy. Once an element was cut from most directions, a sheathing motion can dislodge the segment and pull it into the syringe, enabling it to be pulled out with or without some means of checking that the microparticle is indeed inside it such as measuring the magnetic moment from it, measuring an optical property or other.

An alternative mechanism for cutting a piece of tissue is by introducing a larger hollow tube that surrounds the desired tissue segment and cuts the end by a cutting mechanism such as a mechanical spring that is released by a pulling motion of a thread connected to an external handle. Alternatively, and depending on the tissue type, simply by exerting a pulling force by introducing a vacuum pump or a lower pressure by pulling a syringe.

Cutting can typically be performed in non-essential tissue such as adipose tissue, or in highly regenerative tissue that can withstand the removal of a part of it such as liver tissue. Generally, the device is intended for use in a minimally invasive manner. To avoid harm to patients, the device is intended for use in conjunction with imaging techniques to avoid blood vessels perforation or effects on other sensitive elements.

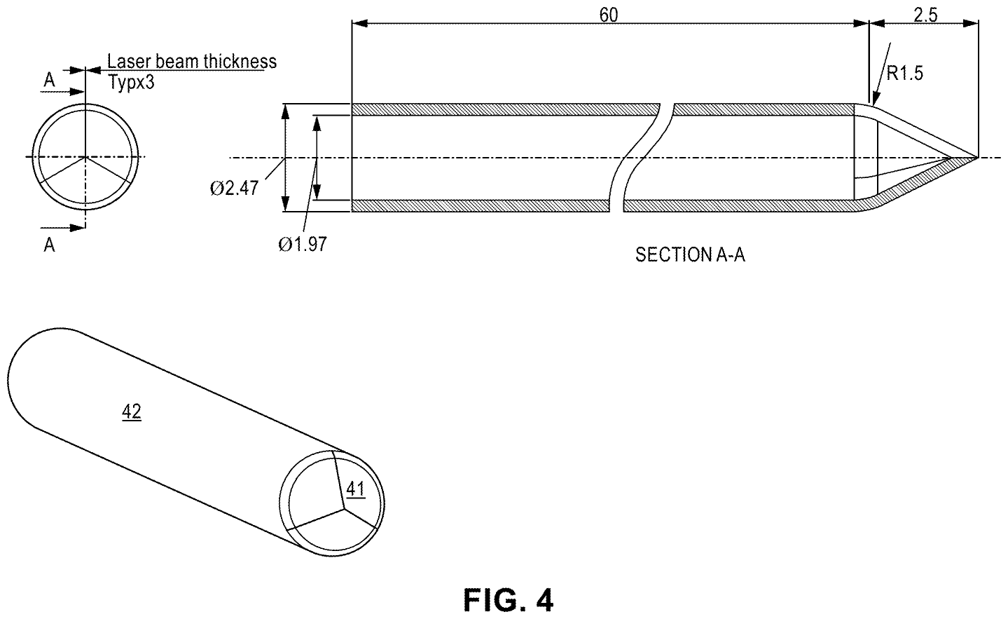

FIG. 4 depicts an embodiment in which an apparatus and method according to the invention are adapted for tissue biopsy from a subject, wherein the apparatus may comprise a tubular needle 42 having cutting leaflets 41 which open and close, cutting through tissue and enclosing the tissue within the tubular needle 42. As shown in FIG. 4, the apparatus may comprise 3 or 4 leaflets, although the number of leaflets is not critical. As depicted, the tip of the apparatus has a length of 2.5 mm, a body length of 60 mm, and inner diameter of 1.97 mm and outer dimeter of 2.47 mm, although these dimensions are for example only and would be expected to vary widely in practice depending on the application without departing from the scope of the invention. The needle may comprise, partly or entirely, a shape memory metal alloy including nickel titanium alloys commonly referred to as Nitinol. Laser cutting a tube of an appropriate size and using a jig to conform the alloy into a desired tip shape allows the formation of a tube with the desired dimensions. The leaflets are sized and shaped to open when pressure is applied from the inside.

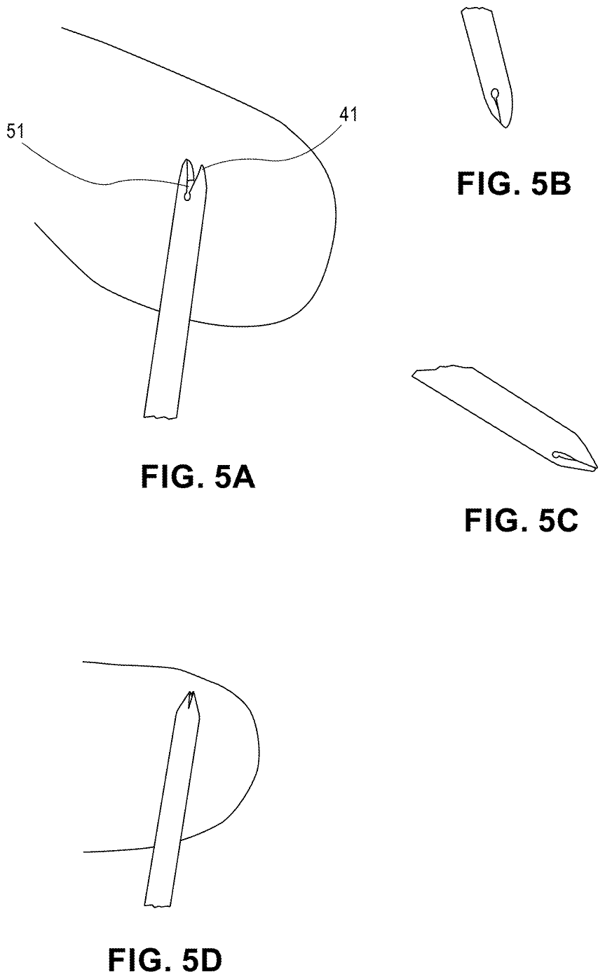

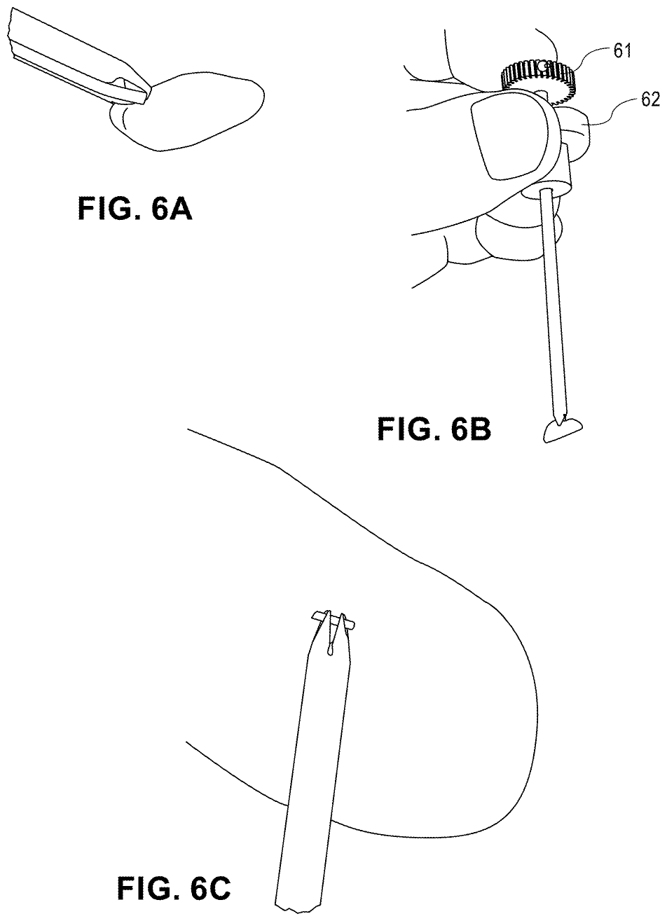

The device may be fitted with an internal rod 51 as the actuating mechanism. The leaflets are normally closed, and after being opened, they tend to elastically apply pressure towards the inside and thus cut through the soft tissue. The leaflets are somewhat sharp due to the laser cutting process. FIG. 5A through FIG. 5D show the opening and closing of the leaflets 41 with the motion of the internal rod 51. The device may be provided with holes (not shown on the drawings) in order to avoid pressure buildup and rupture when the leaflets move.

As depicted in FIG. 6A through FIG. 6C, the device may be fitted with a screw mechanism 61 for the accurate control of the rod's position. The screw is controlled with a knob 62. The device, substantially as depicted, was tested in different media including fresh liver tissue collected from multiple species in order to simulate an in vivo application. The respective retraction flow of a small particle from within the chicken liver is summarized in FIG. 2. The device was fitted with a ferromagnetic internal rod and tested with an external magnetic sensor (e.g., giant magnetoresistance (“GMR”) or tunneling magnetoresistance (“TMR”) device) in order to check the feasibility of magnetic adherence as a modality to enhance the retrieval process in addition to the gripping mechanism.

The above detailed description of the preferred embodiments is not to be considered as limiting the invention, which is defined by the appended claims. Each dependent claim herein sets forth a feature and/or property which may be combined with a feature and/or property described in another dependent or independent claim. The claims should be construed broadly to cover equivalent materials and practices that would be evident to the person of ordinary skill in the art reading the claims in light of the above detailed description.

What is claimed is:

1. An insertion and retraction device for delivery and removal of a microparticle to/from target tissue, comprising:
a magnetic or magnetizable needle or cannula having a distal end, a proximal end and a lumen adapted to convey microparticles;
a tubular catheter receiving the needle or cannula;
a pressure device adapted for delivery of microparticles through the lumen by pressure;
a magnetic field modulator adapted to move the needle or cannula by modulation of a magnetic field; and
a magnetic sensor positioned toward the distal end of the tubular catheter responsive to a magnetic moment of the microparticle.

2. The device according to claim 1, wherein the needle or cannula has a diameter in a range of about 100 to 2000 micron.

3. The device according to claim 2, further comprising a cutting element on the needle or cannula, adapted to cut and remove the target tissue.

4. The device according to claim 1, wherein the distal end of the needle or cannula is steerable within about 1 to 5 mm on either side to guide the distal end to the target tissue.

5. The device according to claim 1, wherein the needle or cannula is comprised of surgical steel, titanium, magnesium, cobalt or chromium, including alloys thereof.

6. The device according to claim 1, wherein the needle or cannula is made from biocompatible polymer selected from polyethylene, polypropylene, polytetrafluoroethylene, polyvinyl chloride, poly methyl methacrylate, polyhydroxy methyl methacrylate, dacron, polyglycolide, polylactic-co-glycolic acid, polylactic acid, polyether ether ketone, polyether sulfone, polyurethane and copolymers and combinations thereof.

7. The device according to claim 5, further comprising a coating on the catheter or needle, wherein the coating is selected from the group consisting of chromium, platinum, palladium, gold and alloys thereof.

8. The device according to claim 5, further comprising a coating on the catheter or needle, wherein the coating is a biodegradable and biocompatible polymer selected from the group consisting of polylactides, polyglycosides, poly(Lactide-co-glycoside), poly(hydroxyalkanoates), perfluorinated or partially fluorinated polymers, polycaprolactones, polypropylene fumarate, polyanhydrides, polyacetals, polycarbonates, polyurethanes, polyphosphazenes and combinations and copolymers thereof.

9. The device according to claim 1, wherein the target tissue is in an organ or within the organ envelope.

10. The device according to claim 1, further comprising a permanent holding magnet adapted to retain and release the microparticle from the distal end of the syringe or catheter.

11. The device according to claim 1, wherein the catheter comprises a radio-opaque element providing visibility to medical imaging equipment.

12. A system for delivery and/or retrieval of a microparticle from target tissue, comprising:
at least one microparticle having a magnetic moment, immobilized in a plug of biocompatible and biodegradable polymer;
a magnetic or magnetizable needle or cannula having a lumen adapted receive and release the at least one microparticle in said plug of biocompatible and biodegradable polymer;
a tubular syringe or catheter adapted to receive the needle or cannula;
a pressure device adapted for delivery of the microparticle through the lumen by pressure;
a magnetic field generator adapted to control movement of the needle or cannula in the syringe or catheter; and
a magnetic sensor positioned toward the distal end of the tubular catheter responsive to the magnetic moment of the microparticle and operatively connected to the magnetic field generator.

13. The system according to claim 12, wherein the plug further comprises: (a) contrasting agent adapted to provide visibility to a permanent holding magnet adapted to retain and release the microparticle from the syringe or catheter; or (b) a therapeutic agent.

14. The system according to claim 12, wherein the microparticle has a longest dimension of about 10 to 2000 micron.

15. The system according to claim 12, further comprising an external mechanical microparticle retrieval element.

16. The system according to claim 12, further comprising an external tissue cutting element.

17. The system according to claim 12, further comprising an external medical imaging system.

18. The device according to claim 12, further comprising a permanent holding magnet adapted to retain and release the microparticle from the distal end of the syringe or catheter in the target tissue.

19. The system according to claim 12, further comprising at least one steering element operatively connected to an external handle, wherein the distal end of the needle or cannula is steerable by said steering element within about 3 to 5 mm on either side of the distal end of the needle to guide the microparticle to the target tissue.

20. The system according to claim 12, wherein the system is adapted to deliver and retrieve said microparticle without removing the needle from the vicinity of the target tissue.

21. An insertion and retraction device for tissue biopsy, comprising:
a magnetic or magnetizable needle or cannula having a distal end, a proximal end and an opening on the distal end adapted to receive a biopsy sample;
leaflets at the distal end of the needle or cannula adapted to open and close the opening;
a rod adapted to be actuated to open and close the leaflets;
a magnetic field modulator adapted to move the needle or cannula by modulation of a magnetic field; and
a magnetic sensor positioned toward the distal end of the tubular catheter responsive to a magnetic moment of the device.

22. The device according to claim 21, wherein the needle or cannula comprises shape memory alloy.

23. The device according to claim 22, wherein the needle or cannula is made of Nitinol.

24. The device according to claim 21, further comprising apertures on the body of the needle or cannula.

25. The device according to claim 21, further comprising a screw mechanism having a knob and being removably connected to the rod for positioning the rod in the lumen of the needle or cannula.

26. The device according to claim 21, wherein the rod is ferromagnetic and adheres to at least one leaflet to facilitate closing of the at least one leaflet.
